(19) United States
(12) Patent Application Publication
(10) Pub. No.: US 2010/0316732 A1
(43) Pub. Date: Dec. 16, 2010

(54) BEVERAGE COMPOSITIONS FOR THE PROMOTION OF JOINT HEALTH IN COMPANION ANIMALS

(76) Inventors: Todd James Smith, Columbus, GA (US); Karen Alexander, Midland, GA (US); Prem Virmani, Columbus, GA (US); David Alexander, (US)

Correspondence Address: Law Offices of Robert F. Zielinski, LLC, 1518 Walnut Street, Suite 1706, Philadelphia, PA, 19102, US

(21) Appl. No.: 12/369,697

(22) Filed: Feb. 11, 2009

Related U.S. Application Data
(60) Provisional application No. 61/028,126, filed on Feb. 12, 2008.

(51) Int. Cl. A61K 33/32; A61P 19/02; A61P 19/04

(52) U.S. Cl. 424/639

(57) ABSTRACT

Aqueous nutritional supplement formulations are described for promoting joint health and for providing a beneficial effect against degenerative joint conditions in companion animals, particularly, dogs and cats by delivering via oral ingestion a nutritional supplement comprising an effective amount of ascorbic acid, suitably bioavailable magnesium and manganese salts and an amino acid in a nutritionally acceptable carrier.

### CROSS REFERENCE TO RELATED APPLICATIONS

This application claims the benefit of Provisional Application No. 61/028,126, filed Feb. 12, 2008, the disclosure of which is incorporated herein by reference to the extent necessary for a full and complete enabling disclosure of this present invention.

### BACKGROUND OF THE INVENTION

### Field of the Invention

The present invention relates generally to a food supplement product and method of use which include a beneficial formulation of ingredients in combination, and which are believed and have been found to have a preventative and remedial effect on connective tissue disorders in companion animals. More particularly, the formulations for the aqueous nutritional supplements herein disclosed are believed and have been found to promote joint health and have a preventative or remedial benefit against joint deterioration in dogs and cats as set forth in greater detail below.

Moreover, the present invention provides a nutritional supplement useful as a dietary food supplement for treating arthritis by oral consumption by companion animals. Said nutritional supplement comprises an effective amount of ascorbic acid, suitably bioavailable magnesium and manganese salts and at least one amino acid in a nutritionally acceptable carrier.

The product and method according to the present invention are particularly advantageous for dogs and cats, although the invention is not so limited. That is, the formulations according to the present invention may have beneficial effects for other companion animals such as horses as well.

The connective tissues of animals are constantly subject to stresses and strains from mechanical forces that can result in afflictions such as osteoarthritis (degenerative joint disease), joint inflammation and stiffness. The majority of degenerative joint disease is the result of mechanical instabilities or aging changes within the joint. This includes old age degenerative arthritis and, in younger animals, may be the result of injuries, bruises, abnormal joint configuration (i.e. hip dysplasia), or mechanical wear from anterior cruciate ligament rupture, patellar luxation, or osteochondritis dissecans. Most of the arthritis occurring in companion animals is osteoarthritis.

Degenerative joint disease is a noninfectious progressive disorder of the weight bearing joints. Increased stress in the joints results in loss of the integrity of the cartilage matrix and the resulting damage causes the accelerated destruction of cartilage components and synovial fluid. The normal articular joint cartilage is smooth, white, and translucent. It is composed of cartilage cells (chondrocytes) imbedded in a sponge-like matrix made of collagen, protein polysaccharides, and water. With early primary arthritis, the cartilage becomes yellow and opaque with localized areas of softening and roughening of the surfaces. As degeneration progresses, the soft areas become cracked and worn, exposing bone under the cartilage. The bone then begins to remodel and increase in density while any remaining cartilage begins to fray. Eventually, osteophytes (spurs of new bone) covered by cartilage form at the edge of the joint. As mechanical wear increases, the cartilage needs repairing. The connective tissues are naturally equipped to repair themselves by manufacturing and remodeling prodigious amounts of collagen (a chief component of connective tissue) and proteoglycans—the other major component of connective tissues; however, with aging, there is a decreased ability to restore and synthesize normal collagen structures the cartilage cells are unable to produce enough of the sponge-like matrix and therefore the damaged cartilage cannot repair itself. The cartilage has no blood supply to enhance healing. This results in pain, deformity and limitation of joint motion.

In dogs, osteoarthritis is a disorder of the synovial joints which is characterized by degeneration of the articular cartilage and by formation of new bone at the joint margins. Hardening of the underlying subchondral bone may also be a feature of osteoarthritis and in some cases, a variable degree of synovial inflammation may be present at some time during the progression of the disease.

Canine Hip Dysplasia (CHD) is a skeletal developmental defect in dogs. A high incidence occurs in larger, rapidly growing dogs or larger working/sporting breeds. The disorder is the result of the upper portion (ball) of the hind legs not fitting properly into the socket of the hip. Dogs are not born with CHD, but as they grow, laxity of muscles and ligaments around the joint in combination with a poor fitness condition produces excess movement at the hip joint. The instability within the joint itself allows the hipbone to pull apart in growth (subluxation).

A general misconception is that CHD is a form of arthritis that affects the hips. Rather severe osteoarthritis is a secondary result of hip dysplasia. It occurs when the bones rub together resulting in irregular bone growth and wear causing osteoarthritis.

It is believed by some, although no substantial proof is given, that CHD is inherited. And many feel the condition is not influenced by diet or caloric intake, but rather is a factor of the animal's overall weight and rapid growth.

Although it cannot be detected at birth, severe cases of CHD may be detectable at six months. The average age for the first symptoms to be displayed is around two years old. Outward signs range from slight to severe pain including difficulty getting up from lying or seated position, climbing stairs, extending back legs, a side to side sway of the crop, a resistance to jumping, a waddling or sway in the gait, lameness especially following exercise, and pushing on the rump may cause the pelvis to drop. Commonly these symptoms are more pronounced on cold, damp days. These are all a result of deterioration of the joint that limits a dog's mobility. CHD may be an inherited trait that is influenced by several genes (polygenic). A licensed veterinarian can properly diagnose CHD through experienced analysis of x-rays of the suspected joint.

Neither the environment nor how a puppy is raised can cause hip dysplasia, however, it could play a role when and perhaps if he/she develops outward symptoms. Some factors that could worsen symptoms of CHD are rough play, jumping, climbing, excess weight gain/rapid growth, calcium supplementation (which increases novel remodeling) or forced distance running especially on tarmac, asphalt or hard surfaces.

The treatment of connective tissue afflictions in animals and concomitant joint heath can be quite problematic. Simply decreasing the stresses to which the connective tissue is subject by isolating or temporarily immobilizing the animal is usually not an option. Consequently, treatment is often directed at controlling the symptoms of the afflictions and not their causes, regardless of the stage of the degenerative process. For example, steroids such as corticosteroids and other anti-inflammatory materials, such as high doses of aspirin are widely used for the treatment of these afflictions in humans. In most cases, these same types of treatment are contraindicated in animals, particularly in dogs. In veterinary medicine, hyaluronic acid and polysulfonated gylcosaminoglycan are used, particularly for equines to reduce connective tissue pain and swelling. While these materials often relieve the pain and swelling associated with maladies arising from connective tissue problems, almost all drugs eventually lose their effectiveness.

There are treatments available for CHD, and some treatments are nonsurgical. However, in many cases, only surgery would help to improve function and reduce pain and inflammation. The administration of drugs such as aspirin, phenylbutazone (“bute”) or NSAIDs and steroids could all be administered to quell pain, but numerous side effects result from oral pain relievers.

Natural products derived from plants and food have frequently been the source of effective drugs, and in recent years there has been an increased interest in the analysis of these natural products, especially where a clinical benefit is claimed. Compounds that have been identified in foods and may be of clinical benefit are the orally administered chondroprotective agents, glucosamine and chondrotin sulphate, which in the body, are normal constituents of articular cartilage. There are studies to suggest that these agents might be effective in humans in the treatment of osteoarthritis. However, there are few reports in the veterinary literature of the clinical efficacy of these oral chondroprotective agents in dogs and other animals. Additionally, glucosamine and chondroitin are not included in the U.S. Food and Drug Administration's lists for approved animal feed additives or ingredients which are generally recognized as safe for inclusion in animal feed.

Nutritional supplements which have however shown some clinical efficacy in the treatment of animal osteoarthritis and CHD are often derived from marine organisms, notably Perna canaliculus (New Zealand Green Lipped Mussel) or hyaluronic acid mucopolysacchadies from animal connective tissue such as rooster combs, bovine tissue sources, bacterial organisms and avian breast bone cartilage. The mode of action of these supplements is not clearly understood at all but the preparation of these supplements typically involves the use of non-readily available or seemingly ‘exotic’ components. As a result their preparation is almost always time consuming and laborious. Additionally, these compounds require encapsulation or incorporation into dry food mixtures as they contain component which are insoluble, not readily miscible or are unstable in dry form or in aqueous solutions. Further still, when incorporation of these supplements into food stuffs is not practical or desirable, these supplements are typically administered in pill or tablet form to the animal which often makes accurate dosing difficult or unpredictable or both.

Flavored aqueous nutritional supplements and waters are also well known for humans and animals. Vitamin waters and sports drinks are generally inappropriate for companion animal consumption because they often contain ingredients that are, at best, superfluous and at worst, potentially harmful for non-humans. Similarly, formulations specifically intended for animals such as dogs, typically contain only electrolytes to replace those which may be lost through animal perspiration and may also contain artificial or naturally derived flavors which are supposed to mimic the taste of certain meats of feed-lot animal parts such as chicken, beef, lamb and liver to increase their appeal to the animal. These formulations fail to address joint health or connective tissue repair. Lastly, many of these beef, lamb, sometimes offal smelling and often fowl flavored waters are highly objectionably odiferous or present a murky appearance which render them organoleptically unacceptable to pet owners, thus decreasing their overall appeal to the purchasing consumer.

There have been other attempts to provide alternative water sources for pets including nutritional fluids for pets which are water-based and which contain vitamins, minerals and other nutrients to use is a supplement for or to replace the drinking water for the pet. Examples of such nutritional drinks for pets are described in U.S. Pat. No. 5,017,389 and PCT Application No. WO 95/28854.

U.S. Pat. No. 5,017,389, issued May 21, 1991, to Green describes a nutritional drink formulated for dogs and other animals containing dextrose, glycine, electrolytes, vitamins and minerals in a buffered pH 7 solution. The nutritional drink is designed to supplement the animal's normal source of nutrition, especially for active dogs and others having increased metabolic rates such as dogs in gestation.

PCT Application No. WO 95/28854 published Nov. 2, 1995, by Duke et al describes a purified drinking water for pets having a tartness and palatability enhancer used to adjust the pH below 5.5. The drinking water preferably also contains sugars, flavoring agents, vitamins, amino acids, electrolytes and minerals.

However for the above formulations, the additional nutritional supplements provided by the prior art formulations are unnecessary and increase the cost of the nutritional fluids such that many pet owners may not utilize these fluids. Additionally, none of the water formulations address the issue of animal joint health.

From the foregoing, it will be appreciated that it would be advantageous to have a nutritional supplement for animals that would promote joint health and provide a beneficial effect against degenerative joint conditions in companion animals.

It will also be appreciated that it would be desirable to have such a nutritional supplement that could be prepared without the use of exotic or esoteric ingredients and which can be prepared with readily available constituents.

It will further be appreciated that it would be desirable to have such a nutritional supplement that would be appealing to both the animal owner and the animal companion.

Finally, it will be appreciated that it would be desirable to have a nutritional supplement that would avoid the shortcomings inherent in the known prior art formulations and provide a stable aqueous orally ingestible formulation that can be easily stored and easily administered to a companion animal.

### BRIEF SUMMARY OF THE INVENTION

It is an object of the present invention to provide a nutritional supplement that promotes joint health and provides preventative or remedial benefits against joint deterioration in companion animals.

It is also an object of the present invention to provide a nutritional supplement which provides a therapeutic composition for the protection and repair of connective tissue in companion animals.

It is a further object of the present invention to provide such a nutritional supplement which includes readily available components capable of providing beneficial therapeutic effects.

It is a further object of the present invention to provide such a nutritional supplement which contains an amino acid and which further contains magnesium, manganese and ascorbic acid for facilitating the repair of connective tissue in animal joints.

It is another object of the present invention to provide such a composition which readily administered and easily absorbed by a companion animal.

In accordance with the teachings of the present invention, disclosed herein is a nutritional supplement capable of the treating and repairing of joint connective tissue animals. The composition includes therapeutic quantities of at least one amino acid such as glycine or proline, in combination with ascorbic acid, magnesium and manganese salts and mixtures thereof.

In further accordance with the teachings of the present invention, disclosed herein is a method for promoting joint health and for the treatment and repairing of connective tissue in companion animals. This method includes the administering of a therapeutically effective quantity of a therapeutic composition including an amino acid such as glycine, in combination with ascorbic acid, magnesium and manganese salts and mixtures thereof.

### DETAILED DESCRIPTION OF THE INVENTION

The present invention is directed to aqueous nutritional supplement formulations for promoting joint health, alleviating arthritic symptoms and for providing a beneficial effect against degenerative joint conditions in companion animals, particularly, dogs and cats, by delivering via oral ingestion an effective amount of at least one amino acid, preferably selected from the group consisting of glycine and/or proline, suitably bioavailable magnesium and manganese salts and ascorbic acid in a nutritionally acceptable carrier.

As used herein, a compound or composition is said to be “acceptable” if its administration can be tolerated by a recipient animal. Such a composition is said to be administered in an “effective amount” if the amount administered is physiologically significant. An agent is physiologically significant if its presence results in technical change in the physiology of a recipient animal. For example, in the promotion of joint health or in alleviation of arthritic symptoms in companion animals, an agent which slows the progression of the disease and/or symptoms or completely treats the disease and/or symptoms, would be considered effective.

Glycine is a non-essential amino acids used to help create muscle tissue and convert glucose into energy. Glycine is used in mammalian animals to help construct normal DNA and RNA strands—the genetic material needed for proper cellular function and formation. Glycine also helps prevent the breakdown of muscle by boosting the body's levels of creatine, a compound that helps build muscle mass. High concentrations of glycine are found not only in the muscles, but in the skin and other connective tissues as well. Almost ⅓ of collagen, which keeps animal skin and connective tissue firm and flexible, is composed of glycine. Without glycine the body would not be able to repair damaged tissues, including connective tissues and wounds would never heal.

The underlying mechanism in promoting joint health and alleviating arthritic symptoms by glycine and proline has been relatively well characterized. It has been shown that both amino acids are precursors in the production of cartilage and have been shown as efficacious in alleviating arthritic symptoms in dogs. Aqueous administration of smaller amino acids such as glycine and proline is preferred as they are more readily absorbed by the small intestine as they are not required to be enzymatically cleaved from proteins or peptides. Such administration results in a more available treatment for osteoarthritic animals and helps address both a cause (regeneration of cartilage and synovial fluid) and effect (anti-inflammatory effects) of osteoarthritis.

Magnesium is the one of the most abundant minerals in the mammalian animal and is essential to good health. Approximately 50% of total body magnesium is found in bone, including cartilage. The other half is found predominantly inside cells of body tissues and organs. Only about 1% of magnesium is found in blood, but the body works very hard to keep blood levels of magnesium constant. Calcium and phosphorus can affect magnesium balance, because high amounts of calcium or phosphorus decrease the absorption of magnesium from the intestines. Additionally, as companion animals age and/or in the presence of certain medications, the ability to absorb magnesium from food declines. Magnesium deficiency in dogs, particular while in the puppy stage, can cause permanent alterations and malformations of joint cartilage and bone which can result in gait alterations, lameness, sub-luxation and hyper-extension of the legs.

Manganese is a mineral that enhances the production of healthy cartilage. It is also essential for the proper development and maintenance of bones and cartilage. Studies suggest that the combination of manganese, Vitamin C, and other compounds such as glucosamine, and chondroitin in joint supplements may reduce the degeneration of cartilage in companion animal arthritic joints. In fact, it has been shown that the interaction of these ingredients works better than any one of them alone. Manganese is essential for a healthy immune system and aids in the production of mucopolysaccharides, which are critical for maintaining healthy joints.

Ascorbic acid mobilizes an animal's self-defense mechanisms that in turn, assist its immune system in overcoming disease. In dogs and cats, ascorbic acid is manufactured in the liver and normally does not need to be supplemented; however, in cases of chronic disease states, such as degenerative joint disease and canine hip dysplasia, ascorbic acid may not be produced in sufficient levels and supplementation is desirable. Ascorbic acid is a powerful antioxidant required to produce collagen, which is the main supportive protein in cartilage, tendon, and connective tissue and is critical to the structure and integrity of healthy joints.

The nutritional supplement of the present invention preferably utilizes a processed water source for the formulation. Preferably still, the water source is distilled water or water purified by reverse osmosis. The water utilized in formulating the flavored nutritional supplement of the present invention should preferably have a total mineral content of less than 200 ppm and should be chlorine free. The presence of higher amounts of minerals and chlorine in the water makes the water less palatable to the companion animals and should be avoided.

The source of the water could be a spring or other natural source having the low levels of dissolved minerals and being essentially free of chlorine or it may be provided by purifying a less suitable source of water such as municipal water to the desired levels of minerals and chlorine. The processed water is preferably prepared by using municipal water and then steam distilling or passing the water through suitable filters including a reverse osmosis filter. The process of purifying the water by this step significantly reduces the amount of dissolved minerals and chlorine to the desired levels. As used herein, the term “water” encompasses water derived from any source, including but not limited to municipal, well, artesian, distilled, filtered, purified and any combinations of the foregoing. Preferably, still the water is potable for animal and/or human consumption.

The present invention may also include flavoring agents to increase the palatability of the nutritional supplement. In such instances, the flavor and odor of the flavored nutritional supplement mimics foods enjoyed by the companion animal or imparts a pleasant aroma familiar to the companion animal's owner. Thus the animal owner is inclined to allow the animal to freely drink the supplement. The flavored nutritional supplement further enhances the health of the animal by providing a clean and pure source of water for the animal which is tasty for the animal and contains the desired amount of the nutritional supplement.

The flavored nutritional supplement of the present invention may also includes flavoring agents which may be natural or artificial and which may mimic certain foods enjoyed by the companion animal. Flavored water formulated for dogs generally contains an artificial beef or lamb flavor while flavored mineral waters formulated for cats may contain an artificial fish, chicken, turkey or liver flavor. A particularly preferred naturally flavoring agent is extract of lemon grass (Cymbopogon citrate), which is further enhanced by the sweetness of the glycine and the tartness of the ascorbic acid.

Cymbopogon is a genus of about 55 species of grasses, native to warm temperate and tropical regions of the Old World and Oceania. It is a tall perennial grass. Common names include lemon grass, lemongrass, barbed wire grass, silky heads, citronella grass, fever grass or Hierba Luisa amongst many others

In addition to providing a pleasant aroma and taste for the consumer (i.e., the companion animal), lemon grass extract also imparts health benefits in various ways. Lemon grass extract has been shown to exhibit antimicrobial and antifungal properties. Lemon grass has also been reported to improve cardiovascular health in mammals and is also a natural insect repellent. In addition, lemon grass extract has been reported as being used both as an analgesic and mood altering and calming supplement in nutritional supplements.

It will be appreciated that other plant extracts with similar or different beneficial properties are also contemplated by the present invention. In particular, several plant-based polyphenolic compounds have been reported to possess health benefits. Several dietary sources, including herbs and spices, fruits and vegetables, and tea and wine, contain an array of biologically active compounds that have been shown to be effective in retarding oxidation of low-density lipoproteins (LDL) and promoting vascular relaxation. Organic extracts enriched in polyphenols prepared from palm fronds (Elaesis guineensis); lemon grass (Cymbopogon citrates); ginger root (Zingiber officinale), papaya shoots (Carica papaya) and green chilli (Capsicum frutescenes) may also be used as flavoring agents.

Preferably still, the amount of the flavoring agents added to the nutritional supplement should be sufficient to provide an adequate level of olfactory stimulus to the animal but not so much as to overpower the animal while at the same time providing a pleasant aroma for the animal companion's owner. For many of the commercially available flavors this will generally be between about 15 and 1000 ppm of the concentrated flavor with a preferred level being approximately 20 to 200 ppm. This range may vary however depending upon the nature and concentration of the flavoring agents provided by the manufacturer.

Additionally, flavored nutritional supplement with only minimal amounts of glycerin have been generally found to be more palatable to the animals and increases the likelihood of the animal ingesting an adequate amount of the water for their daily requirements. By using only small amounts of glycerin, the clarity of the water is also improved as there are reduced organic materials and no film or residue which is observed with waters containing high amounts of glycerin, thus making it appealing to the animal companion owner.

In addition to the flavoring agents, the nutritional supplement of the present invention may also contain certain other essential minerals for the animal's health. For example, for dogs, the formulation may contain other trace minerals such as phosphorus in an amount of approximately 50 ppm while for cats the mineral water may contain phosphorus at about 50 ppm, and calcium at about 50 ppm.

To provide the adequate levels of phosphorous a soluble phosphorous containing compound is utilized. The phosphorous containing compound is preferably a phosphorous containing salt such as calcium phosphate, sodium pyrophosphate, sodium acid pyrophosphate, sodium polyphosphate, and tetrasodium pyrophosphate among others. The preferred phosphorous containing compound is selected from calcium phosphate, sodium pyrophosphate and sodium acid pyrophosphate. In some embodiments, the use of calcium phosphate also provides the proper level of calcium in the water. [0049] In other embodiments of the nutritional supplement of the present invention a minimal amount of carbohydrates and other organic materials may be used. Formulations which are low in carbohydrates may be less capable of supporting growth of bacteria and other microbial contaminants. In these embodiments, it has been found that relatively low levels of preservatives are necessary for the nutritional supplement of the present invention. The low level of preservatives may also make the nutritional supplement more palatable to the companion animals as higher levels of commonly used preservatives have an objectionable aftertaste.

One example of the flavored nutritional supplement of the present invention is formulated by adding the constituents to the purified water and mixing as set forth in Table 1 for the small batch process. The phosphorous containing compound and optionally the magnesium containing compound and other additives are then added, the water mixed, the pH adjusted if required and dispensed into suitable containers.

### Example 1

### Canine Water Ingredients

### Joint Health Formula

| | | | Maximum |
|---|---|---|---|
| Ingredient | Function | Milligrams/Liter | Solubility/Liter |
| Water | Provides hydration | q.s. to 1 liter | Not Applicable |
| | and acts as a vehicle | | |
| | for the added vitamins | | |
| | and/or minerals. | | |
| Sodium | Preservative | 350 | See Note 1 |
| Hexametaphosphate | | | |
| Potassium Sorbate | Preservative | 250 | See Note 1 |
| Sodium Benzoate | Preservative | 100 | See Note 1 |
| Phosphoric Acid | Decreases the pH to | 366.4 of 100% OR | See Note 2 |
| | minimize microbial | 458.0 of 75% | |
| | growth. | | |
| Potassium Citrate | Buffers the pH | 175 | See Note 2 |
| Natural Flavors | Provides an appealing | 240 | See Note 3 |
| | taste. | | |
| Glycine | A building block of | 250 | 100 grams/liter |
| | collagen. Also a | | See Note 7 |
| | precursor to the | | |
| | formation of natural | | |
| | condroitin. | | |
| Ascorbic Acid (Vitamin | An antioxidant | 100 | 3.3 grams/liter |
| C) | important in the | | See Note 6 |
| | synthesis of collagen | | |
| | and cartilage. | | |
| Manganese citrate | Helps with protein | 0.3 | 1000 mg/liter |
| | synthesis and | | See Note 5 |
| | providing stability to | | |
| | muscle and nerve cell | | |
| | membranes. | | |
| Magnium chloride | Helps in bone and | 10 es | 1100 mg/liter |
| | cartilage development. | | See Note 4 |

Note 1 These ingredients are added as preservatives and for control of microbial growth. The levels selected are deemed safe for ingestion by dogs, cats and humans. Neither higher nor lower levels can be recommended while continuing to ensure control of bacteria and/or fungi while ensuring no adverse effects upon animal safety. Preservatives can be eliminated with use of other methods of microbial control (e.g. hot fill; aseptic fill; pasteurization; sterilization by heat or filtration).
Note 2 These ingredients are added to provide an optimum pH for the functioning of the preservatives. These ingredients can be eliminated if other means for microbial control are implemented.
Note 3 The amount of natural flavor added is completely dependent upon the type and strength of flavor chosen. The flavor level selected for this formula was chosen based upon testing conducted at the humane society with dogs and in the lab with humans to create a flavor and intensity that was appealing to both dogs and their owners.
Note 4 While the maximum levels of solubility have been determined for the 4 active ingredients, it must be noted that these are not necessarily safe levels for ingestion by dogs. While there is virtually no information on Magnesium toxicity in dogs, the NRC has stated concentrations of 0.8-1.7 grams/kilogram of food is likely to be safe. If an adult dog's diet contained no other magnesium, the level in this formula could be increased by 80 times and still be in the safe range if the dog ingested 1 liter of water in place of 1 kg of food.
Note 5 While the maximum levels of solubility have been determined for the 4 active ingredients, it must be noted that these are not necessarily safe levels for ingestion by dogs. There is no data available to predict the safe upper limit of Manganese in dogs. The NRC recommends 80 micrograms/kilogram of body weight/day. Current formula has 300 micrograms/liter. Based upon these numbers, a 35 pound/77 kg beagle would require approximately 6160 micrograms/day. 6000 micrograms is equivalent to 6 milligrams which is approximately 20 times what is in one liter of our current formula, assuming there is no other source of Mn provided to the dog.
Note 6 Vitamin C is considered a non-essential nutrient and therefore, required and toxic levels have not been identified in dogs. Several studies with dogs have shown no adverse effects at levels of 2.5, 3.0 and 9.0 grams/day. Given the maximum solubility is approximately 3.3 grams/liter, a dog could theoretically ingest up to 3 liters without adverse effects.
Note 7 Glycine is considered a non-essential nutrient in dogs and cats and based on dietary intake of protein can be administered in amount up to 50 times this amount without any ill effect. Proline is also a non-essential nutrient in dogs and cats and can be administered in place of or together with glycine within the preferred ranges.

It will be appreciated that the preferred levels of the constituent ingredients may be adjusted upwardly and downwardly by as much as 10 to 90% and depending on the preparation techniques, some ingredients may be eliminated entirely without departing from the spirit of the invention.

TABLE 1
Canine Lemongrass Water
1 Liter Mix Instructions

| | | |
|---|---|---|
| Water | 990.78 | mL |
| Preservatives solution | 4.2 | grams |
| Mg, Mn and Glycine | 2.11 | grams |
| Buffering agents | 3.98 | grams |
| Ascorbic Acid*** | 1.20 | grams |
| Flavor (lemon grass) | 2.0 | mL |
| Syrup Yield | 1.0 | Liter |

### Ingredients Added in Order According to Mixing Instructions

### Directions:

1 Meter hot treated water (110-120 degrees F.) into mixing tank.
2 While mixing add preservatives and a prepared combination of manganese, magnesium and glycine. Allow materials to dissolve completely while continuing to agitate.
3 Slowly add buffering agents and mix thoroughly. Continue to agitate.
4 Add ascorbic acid and dissolve completely.
5 While mixing, slowly add remaining ingredients, and dissolve completely.
6 Add additional ingredients as necessary, and agitate until completely blended.
7 Before bottling, allow sufficient time to expel entrapped air.
8 Check pH and strain prior to packaging.

The resulting nutritional supplement of the present invention prepared in accordance with the ingredients and directions in Table 1 is a clear, pleasant tasting drinking water that is infused with the flavor and aroma of lemon grass. Companion animals readily consume the flavored nutritional supplement and companion animal owners find the lemon grass aroma to be preferable to those formulations which have strong or objectionable odors.

The suggested administration of the nutritional supplements herein are expressed in effective amounts of active ingredient and will, of course, vary depending upon known factors such as the pharmacodynamic characteristics of the particular active ingredient and its concentration in solution and amount of administration; the age, sex, health and weight of the companion animal; nature and extent of symptoms; kind of concurrent treatment, frequency of treatment and the effect desired.

Although the invention has been described in detail with reference to certain preferred embodiments, those skilled in the art will recognize that the invention can be practiced with variations and modifications within the scope and spirit of the invention as described and defined in the following claims.

What is claimed is:

1. A nutritional supplement for promotion of joint health and repair of connective tissue in companion animals comprising therapeutically effective quantities of an amino acid selected from the group consisting of glycine and proline, magnesium, manganese and mixtures thereof in combination with ascorbic acid in a nutritionally acceptable carrier.

2. The nutritional supplement of claim 1 further comprising a flavoring agent.

3. The nutritional supplement of claim 2 wherein the flavoring agent is selected from the group consisting of natural and artificial flavors.

4. The nutritional supplement of claim 2 wherein the amino acid is glycine.

5. The nutritional supplement of claim 1 wherein the nutritionally acceptable carrier is water.

6. A method for promoting joint health and relieving discomforts associated with joint disorders in a companion animal comprising the steps of delivering to said animal by oral ingestion a nutritional supplement comprising therapeutically effective quantities of an amino acid selected from the group consisting of glycine and proline, magnesium, manganese and mixtures thereof in combination with ascorbic acid in a nutritionally acceptable carrier.

7. The method of claim 6 further comprising the step of adding a flavoring agent to the carrier, and wherein the carrier comprises water.

8. The method of claim 7 wherein the nutritional supplement is provided to the companion animal to consume as desired.

9. The method of claim 8 wherein the companion animal is an equine, canine, or feline species.

10. The method of claim 6 wherein the joint disorder is the result of an arthritic condition.

11. The method of claim 10 wherein the arthritic condition is osteoarthritis.

12. The method of claim 10 wherein the joint disorder is the result of an inflammatory condition involving skeletal or musculoskeletal structures.

13. A nutritional supplement consisting essentially of a therapeutically effective quantities of glycine, magnesium, manganese and mixtures thereof in combination with ascorbic acid in a nutritionally acceptable carrier and a flavoring agent, the nutritional supplement provided in an orally ingestible dosage form.

14. The nutritional supplement of claim 13 wherein the orally ingestible dosage form is an aqueous form.
